(19) United States
(12) Patent Application Publication
(10) Pub. No.: US 2020/0297829 A1
(43) Pub. Date: Sep. 24, 2020

(54) Integrative Immunotherapy for Cancer Treatment

(71) Applicant: BATU BIOLOGICS, INC., San Diego, CA (US)

(72) Inventors: Samuel C. Wagner, San Diego, CA (US); Thomas E. Ichim, San Diego, CA (US)

(21) Appl. No.: 16/356,892

(22) Filed: Mar. 18, 2019

(51) Int. Cl. A61K 39/00; A61P 35/00; A61K 39/39

(52) U.S. Cl. CPC A61K 39/0011; G16B 20/00; A61K 39/39; A61P 35/00

(57) ABSTRACT

Disclosed are means, methods, and compositions of matter useful for treatment of cancer through integrated immunotherapy. In one embodiment a synergistic protocol is comprised of: a) establishing a proteomic, genomic, and immunological profile of the patient; b) stimulation of a change in the tumor microenvironment in order to modify the tumor microenvironment to be more conducive to immunotherapy; c) priming the immune system in order to be prepared for immune activation; d) activate vaccination in order to induce antigen specific immune responses; e) induction of damage to the tumor in an immunogenic manner; f) immune focusing by utilization of epitope specific vaccination; and g) boosting the immune response.

### CROSS-REFERENCE TO RELATED APPLICATIONS

This patent application takes priority from Provisional Patent Application No. 62/644,226, titled Integrative Immunotherapy for Cancer Treatment, filed on Mar. 16, 2018, the contents of which are expressly incorporated herein by this reference as though set forth in their entirety and to which priority is claimed.

### FIELD OF THE INVENTION

The invention pertains to the field of cancer therapeutics, more particularly, the field pertains to the use of combination therapies in the treatment of cancer, more particularly, the invention pertains to a multi-disciplinary attack on cancer by integration of several cancer therapies with the objective of obtaining a synergistic effect.

### BACKGROUND OF THE INVENTION

The treatment of cancer has progressed significantly with the development of pharmaceuticals that more efficiently target and kill cancer cells. To this end, researchers have taken advantage of cell-surface receptors and antigens selectively expressed by cancer cells to develop drugs based on antibodies that bind the tumor-specific or tumor-associated antigens. The concept that the immune system plays a role in controlling oncogenesis was described in the 1960s by Burnet, who noticed higher levels of malignancies in patients that suffer from inborn immunodeficiences. This has subsequently been verified over the decades in a variety of neoplasias, and in a variety of immune deficiencies. An example of the impact that congenital immunodeficiency has on incidence of cancer can be seen in a study of 377 patients with primary hypogammaglobulinaemia, mainly common variable immunodeficiency (CVID), 316 patients survived the first 2 years after diagnosis and were the subject of a study of cancer incidence. Among the 220 patients with CVID, there was a 5-fold increase of cancer due mainly to large excesses of stomach cancer (47-fold) and lymphomas (30-fold). The excess of stomach cancer is probably related to the high frequency of achlorhydria in CVID. 3 of the 7 patients with stomach cancer and CVID survived for 5 years or longer. In another study, a 30-fold increase in incidence of colorectal cancer was observed in patients suffering from X-linked agammaglobulinaemia (XLA). Interestingly, in patients with CVID, the patients that have lower CD8 T cell numbers and activity are more susceptible to cancer development. Immune recognition of cancers is further supported by studies in which infiltration of tumors by lymphocytes is associated with enhanced survival. This has been demonstrated in many tumor types, for example in breast, gallbladder, and ovarian.

Thus, there remains a need for development of pharmaceuticals that more efficiently target and kill cancer cells and that seek to minimize the issues that exist with currently available drug conjugate compositions. There also remains a need for methods of using such specifically targeted pharmaceuticals to treat human diseases associated with cell proliferation, such as cancer.

### DESCRIPTION OF THE INVENTION

The invention teaches the identification of cancer specific properties, the utilization of these properties to generate tumor specific immune responses but enabling the tumor specific immune response by augmenting cancer exposure to immune system by selective killing of tumor endothelial cells using ValloVax or other means of vaccination. In order to augment immunity first the patient's blocking factors are removed by immunopheresis means, subsequently the immune system of the patient is primed by administration of agents that increase dendritic cell numbers to the site of vaccination, subsequently, the dendritic cells are matured by administration of maturation agents. In order to induce immunity ValloVax, or cancer specific peptides are administered together with adjuvants. Alternatives include administration of dendritic cells that are pulsed with antigens. Additionally, tumor cell immunogenic death is induced in order to provide endogenous antigens. Focusing of immunity is achieved by administration of peptides that represent the tumor. This provides a means of selectively amplifying the immune response only to the tumor. Once this immune response is activated, the proliferating clones are expanded by use of booster procedures such as IL-2 administration, administration of immunological checkpoint inhibitors, or IL-7.

“Adjuvant” refers to a substance that is capable of enhancing, accelerating, or prolonging an immune response when given with a vaccine immunogen.

“Agonist” refers to is a substance which promotes (induces, causes, enhances or increases) the activity of another molecule or a receptor. The term agonist encompasses substances which bind receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells.

The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention.

Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma.

In the context of the present invention the term “culturing” refers to the in vitro propagation of cells or organisms in media of various kinds. It is understood that the descendants of a cell grown in culture may not be completely identical (morphologically, genetically, or phenotypically) to the parent cell. A suitable culturing medium can be selected by the person skilled in the art and examples of such media are RPMI medium or Eagles Minimal Essential Medium (EMEM).

The terms “vaccine”, “immunogen”, or immunogenic composition” are used herein to refer to a compound or composition that is capable of conferring a degree of specific immunity when administered to a human or animal subject. As used in this disclosure, a “cellular vaccine” or “cellular immunogen” refers to a composition comprising at least one cell population, which is optionally inactivated, as an active ingredient. The immunogens, and immunogenic compositions of this invention are active, which mean that they are capable of stimulating a specific immunological response (such as an anti-tumor antigen or anti-cancer cell response) mediated at least in part by the immune system of the host. The immunological response may comprise antibodies, immunoreactive cells (such as helper/inducer or cytotoxic cells), or any combination thereof, and is preferably directed towards an antigen that is present on a tumor towards which the treatment is directed. The response may be elicited or restimulated in a subject by administration of either single or multiple doses.

A compound or composition is “immunogenic” if it is capable of either: a) generating an immune response against an antigen (such as a tumor antigen) in a naive individual; or b) reconstituting, boosting, or maintaining an immune response in an individual beyond what would occur if the compound or composition was not administered. A composition is immunogenic if it is capable of attaining either of these criteria when administered in single or multiple doses.

The term “T-cell response” means the specific proliferation and activation of effector functions induced by a peptide in vitro or in vivo. For MHC class I restricted cytotoxic T cells, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, preferably granzymes or perforins induced by peptide, or degranulation.

The term “peptide” is used herein to designate a series of amino acid residues, connected one to the other typically by peptide bonds between the alpha-amino and carbonyl groups of the adjacent amino acids. The peptides are preferably 9 amino acids in length but can be as short as 8 amino acids in length, and as long as 10, 11, 12, or even longer, and in case of MHC class II peptides (e.g. elongated variants of the peptides of the invention) they can be as long as 15, 16, 17, 18, 19, 20 or 23 or more amino acids in length.

Furthermore, the term “peptide” shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoro-acetate) salts. It has to be noted that the salts of the peptides according to the present invention differ substantially from the peptides in their state(s) in vivo, as the peptides are not salts in vivo.

The term “peptide” shall also include “oligopeptide”. The term “oligopeptide” is used herein to designate a series of amino acid residues, connected one to the other typically by peptide bonds between the alpha-amino and carbonyl groups of the adjacent amino acids. The length of the oligopeptide is not critical to the invention, as long as the correct epitope or epitopes are maintained therein. The oligopeptides are typically less than about 30 amino acid residues in length, and greater than about 15 amino acids in length.

The term “polypeptide” designates a series of amino acid residues, connected one to the other typically by peptide bonds between the alpha-amino and carbonyl groups of the adjacent amino acids. The length of the polypeptide is not critical to the invention as long as the correct epitopes are maintained. In contrast to the terms peptide or oligopeptide, the term polypeptide is meant to refer to molecules containing more than about 30 amino acid residues.

A peptide, oligopeptide, protein or polynucleotide coding for such a molecule is “immunogenic” (and thus is an “immunogen” within the present invention), if it is capable of inducing an immune response. In the case of the present invention, immunogenicity is more specifically defined as the ability to induce a T-cell response. Thus, an “immunogen” would be a molecule that is capable of inducing an immune response, and in the case of the present invention, a molecule capable of inducing a T-cell response. In another aspect, the immunogen can be the peptide, the complex of the peptide with MHC, oligopeptide, and/or protein that is used to raise specific antibodies or TCRs against it.

Cancer-testis antigens: The first TAAs ever identified that can be recognized by T cells belong to this class, which was originally called cancer-testis (CT) antigens because of the expression of its members in histologically different human tumors and, among normal tissues, only in spermatocytes/spermatogonia of testis and, occasion-ally, in placenta. Since the cells of testis do not express class I and II HLA molecules, these antigens cannot be recognized by T cells in normal tissues and can therefore be considered as immunologically tumor-specific. Well-known examples for CT antigens are the MAGE family members and NY-ESO-1. Differentiation antigens: These TAAs are shared between tumors and the normal tissue from which the tumor arose. Most of the known differentiation antigens are found in melanomas and normal melanocytes. Many of these melanocyte lineage-related proteins are involved in biosynthesis of melanin and are therefore not tumor specific but nevertheless are widely used for cancer immunotherapy. Examples include, but are not limited to, tyrosinase and Melan-A/MART-1 for melanoma or PSA for prostate cancer. Over-expressed TAAs: Genes encoding widely expressed TAAs have been detected in histologically different types of tumors as well as in many normal tissues, generally with lower expression levels. It is possible that many of the epitopes processed and potentially presented by normal tissues are below the threshold level for T-cell recognition, while their over-expression in tumor cells can trigger an anticancer response by breaking previously established tolerance. Prominent examples for this class of TAAs are Her-2/neu, survivin, telomerase, or WT1. Tumor-specific antigens: These unique TAAs arise from mutations of normal genes (such as .beta.-catenin, CDK4, etc.).

Some of these molecular changes are associated with neoplastic transformation and/or progression. Tumor-specific antigens are generally able to induce strong immune responses without bearing the risk for autoimmune reactions against normal tissues. On the other hand, these TAAs are in most cases only relevant to the exact tumor on which they were identified and are usually not shared between many individual tumors. Tumor-specificity (or -association) of a peptide may also arise if the peptide originates from a tumor- (-associated) exon in case of proteins with tumor-specific (-associated) isoforms. TAAs arising from abnormal posttranslational modifications: Such TAAs may arise from proteins which are neither specific nor overexpressed in tumors but nevertheless become tumor associated by posttranslational processes primarily active in tumors. Examples for this class arise from altered glycosylation patterns leading to novel epitopes in tumors as for MUC1 or events like protein splicing during degradation which may or may not be tumor specific. Oncoviral proteins: These TAAs are viral proteins that may play a critical role in the oncogenic process and, because they are foreign (not of human origin), they can evoke a T-cell response. Examples of such proteins are the human papilloma type 16 virus proteins, E6 and E7, which are expressed in cervical carcinoma. T-cell based immunotherapy targets peptide epitopes derived from tumor-associated or tumor-specific proteins, which are presented by molecules of the major histocompatibility complex (MHC). The antigens that are recognized by the tumor specific T lymphocytes, that is, the epitopes thereof, can be molecules derived from all protein classes, such as enzymes, receptors, transcription factors, etc. which are expressed and, as compared to unaltered cells of the same origin, usually up-regulated in cells of the respective tumor.

Therefore, TAAs are a starting point for the development of a T cell-based therapy including but not limited to tumor vaccines. The methods for identifying and characterizing the TAAs are usually based on the use of T-cells that can be isolated from patients or healthy subjects, or they are based on the generation of differential transcription profiles or differential peptide expression patterns between tumors and normal tissues. However, the identification of genes over-expressed in tumor tissues or human tumor cell lines, or selectively expressed in such tissues or cell lines, does not provide precise information as to the use of the antigens being transcribed from these genes in an immune therapy. This is because only an individual subpopulation of epitopes of these antigens are suitable for such an application since a T cell with a corresponding TCR has to be present and the immunological tolerance for this particular epitope needs to be absent or minimal. In a very preferred embodiment of the invention it is therefore important to select only those over- or selectively presented peptides against which a functional and/or a proliferating T cell can be found. Such a functional T cell is defined as a T cell, which upon stimulation with a specific antigen can be clonally expanded and is able to execute effector functions (“effector T cell”).

For the purpose of vaccine production, the cancer cells are isolated from an autologous subject, meaning that they will be used to treat the same subject from whom they were derived. Alternatively, the cancer cells could be used in an HLA-matched heterologous subject. Typically, the cells are isolated during a biopsy procedure or during surgical tumor removal. The cancer cells may be derived from any type of malignancy and, in an aspect, they are derived from lung cancer, including small cell lung cancer and non-small cell lung cancer (e.g. adenocarcinoma), pancreatic cancer, colon cancer (e.g. colorectal carcinoma, such as, for example, colon adenocarcinoma and colon adenoma), esophageal cancer, oral squamous carcinoma, tongue carcinoma, gastric carcinoma, liver cancer, nasopharyngeal cancer, hematopoietic tumors of lymphoid lineage (e.g. acute lymphocytic leukemia, B-cell lymphoma, Burkitt's lymphoma), non-Hodgkin's lymphoma (e.g. mantle cell lymphoma), Hodgkin's disease, myeloid leukemia (for example, acute myelogenous leukemia (AML) or chronic myelogenous leukemia (CML)), acute lymphoblastic leukemia, chronic lymphocytic leukemia (CLL), thyroid follicular cancer, myelodysplastic syndrome (MDS), tumors of mesenchymal origin, soft tissue sarcoma, liposarcoma, gastrointestinal stromal sarcoma, malignant peripheral nerve sheath tumor (MPNST), Ewing sarcoma, leiomyosarcoma, mesenchymal chondrosarcoma, lymphosarcoma, fibrosarcoma, rhabdomyosarcoma, melanoma, teratocarcinoma, neuroblastoma, brain tumors, medulloblastoma, glioma, benign tumor of the skin (e.g. keratoacanthoma), breast carcinoma (e.g. advanced breast cancer), kidney carcinoma, nephroblastoma, ovary carcinoma, cervical carcinoma, endometrial carcinoma, bladder carcinoma, prostate cancer, including advanced disease and hormone refractory prostate cancer, testicular cancer, osteosarcoma, head and neck cancer, epidermal carcinoma, multiple myeloma (e.g. refractory multiple myeloma), or mesothelioma. In an aspect, the cancer cells are derived from a solid tumor.

Typically, the cancer cells are derived from a breast cancer, colorectal cancer, melanoma, ovarian cancer, pancreatic cancer, gastric cancer, or prostate cancer. More typically, the cancer cells are derived from a prostate cancer. While most cancer cells do not naturally express much if any MHCII on their cell surface, it will be understood that if the cancer cells are derived from antigen-presenting cells, such as a B cell cancer for example, these cells may already express MHCII on their cell surface. It is contemplated that unmodified cancer cells that already express MHCII could be explicitly excluded from the present invention. In other words, it is contemplated that the present invention could encompass cancer cells that are MHCII-negative, MHCII-positive, or both prior to modification according to the present invention. Alternatively, such cells could be included in the invention and it will be understood that, since these cells already express MHCII, incubation with an MHCII-inducing agent is merely optional in order to increase the level of expression. For example, the MHCII-inducing agent may be IFN-.gamma., or it may be an MHCII expression vector that is used to transfect or transduce the cancer cells. The MHCII-inducing agent also encompasses a cell expressing MHCII, in that cells that express MHCII could be fused via cell fusion with the cancer cells to render the cancer cells MHCII positive. Examples of such cells include B cells, dendritic cells, macrophages, and monocytes. In another aspect, the MHCII inducing agent may be an agent that activates the MHCII transactivator (CIITA) sequence.

In one embodiment the invention provides a means of generating a population of cells with tumoricidal ability. 50 ml of peripheral blood is extracted from a cancer patient and peripheral blood monoclear cells (PBMC) are isolated using the Ficoll Method. PBMC are subsequently resuspended in 10 ml STEM-34 media and allowed to adhere onto a plastic surface for 2-4 hours. The adherent cells are then cultured at 37° C. in STEM-34 media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment the generated DC are used to stimulate T cell and NK cell tumoricidal activity. Specifically, generated DC may be further purified from culture through use of flow cytometry sorting or magnetic activated cell sorting (MACS) or may be utilized as a semi-pure population. DC may be added into the patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to the T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel.

In one embodiment, the invention provides a means for decreasing immunological tolerance, or in some cases breaking immunological tolerance, through the use of immunopheresis. Tolerance may be defined as a selective immunological ignorance of a specific tissue. Numerous examples of tolerance are known in the prior art, for example in pregnancy, cancer, transplantation, and successfully treated autoimmune conditions. In one specific embodiment the invention teaches the use of methods to remove soluble TNF-alpha receptors in a cancer patient. Through the removal of these proteins, the invention teaches, an “anti-tolerogenic”effect is induced, which allows for enhanced ability to immunize against tumor antigens. The reduction of soluble TNF-alpha receptors is performed within the context of the invention for a period of time sufficient to induce immune activation, without inducing chronic inflammation. Indeed, it is known that TNF-alpha produced by the interaction between tumor cells and immune cells, may be assist in tumor growth and other characteristics of cancer such as cachexia. Additionally, it is known that inhibition of TNF-alpha in patients exposed to chronic inflammation, such as patients with rheumatoid arthritis, actually have less cancer incidence, not more.

In one embodiment of the invention, immunopheresis is performed to reduce levels of circulating TNF-alpha soluble receptors in order to allow more efficient killing of tumors by radiation, and by combination of radiation and dendritic cell therapy. Indeed, previous studies have shown ability of radiation alone, or combined with dendritic cell therapy to induce increased production of TNF-alpha.

In some embodiments of the invention, immunization is performed through induction of localized tumor cell death. In one embodiment, immunopheresis is performed together with isolated limb perfusion of TNF-alpha, means of performing this procedure are known in the art and described in the following references. Through inducing local cellular death of neoplastic tissue, resulting endogenous antigens are released which serve as an autologous vaccine.

In one embodiment of the invention immunization is performed prior to immunopheresis, with subsequent activation of immunity through reduction of circulating TNF-alpha soluble receptor by immunopheresis. This reduction leads to activation of innate immunity, which serves as the basis for enhancement of tumor immunogenicity. Further enhancement of immunogenicity can be performed by combination with inhibitors of immune inhibitor molecules. The immune inhibitory molecules are referred to as checkpoint inhibitors and include PD1, PD-L1, CTLA4, TIM3, LAGS, VISTA, BTLA, TIGIT, LAIR1, CD160, 2B4 and TGFR beta. Inhibition of an inhibitory molecule, e.g., by inhibition at the DNA, RNA or protein level, can lead to increased immune function, as described herein. In embodiments, an inhibitory nucleic acid, e.g., an inhibitory nucleic acid, e.g., a dsRNA, e.g., an siRNA or shRNA, can be used to inhibit expression of an inhibitory molecule. In an embodiment the inhibitor is an shRNA. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101 and marketed as Yervoy™; Bristol-Myers Squibb; Tremelimumab (IgG2 monoclonal antibody available from Pfizer, formerly known as ticilimumab, CP-675,206).). In an embodiment, the agent is an antibody or antibody fragment that binds to TIM3. In an embodiment, the agent is an antibody or antibody fragment that binds to LAG3.

In some embodiments adjuvants to exogenous vaccines are utilized, the adjuvant may be selected from monophosphoryl Lipid A/synthetic trehalose dicorynomycolate (MPL-TDM), AS021/AS02, nonionic block co-polymer adjuvants, CRL 1005, aluminum phosphates, AIPO4), R-848, imiquimod, PAM3CYS, poly (I:C), loxoribine, bacille Calmette-Guerin (BCG), Corynebacterium parvum, CpG oligodeoxynucleotides (ODN), cholera toxin derived antigens, CTA 1-DD, lipopolysaccharide adjuvants, complete Freund's adjuvant, incomplete Freund's adjuvant, saponin, mineral gels, aluminum hydroxide, surface active substances, lysolecithin, pluronic polyols, polyanions, peptides, oil or hydrocarbon emulsions in water, MF59, Montanide ISA 720, keyhole limpet hemocyanins (KLH), dinitrophenol, and combinations thereof.

In some embodiments, the culture of the cells is performed by starting with purified lymphocyte populations, for example, the step of separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used.

In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-gamma, transforming growth factor (TGF)-beta, IL-15, IL-7, IFN-alpha, IL-12, CD40L, and IL-27.

From the viewpoint of enhancing cellular immunity, particularly suitably, IL-2, IFN-gamma, or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method.

In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

The invention provides means of utilizing endothelial progenitor cells and products derived from the endothelial progenitor cells as a cancer vaccine which selectively induces immunity towards tumor vasculature and not healthy, non-malignant, vasculature. In one embodiment the invention teaches the utilization of culture conditions which mimic the tumor microenvironment as a means of creating a cellular population that resembles tumor endothelial cells. Culture conditions include the growth of endothelial progenitor cells in acidic conditions which resemble the tumor microenvironment. Numerous papers have characterized the acidic conditions in the tumor microenvironment and are incorporated by reference.

Interestingly, tumor acidic conditions are believed to be associated with resistance to immunotherapy. In a recent study it was shown that an acidic pH environment blocked T-cell activation and limited glycolysis in vitro. IFNγ release blocked by acidic pH did not occur at the level of steady-state mRNA, implying that the effect of acidity was posttranslational. Acidification did not affect cytoplasmic pH, suggesting that signals transduced by external acidity were likely mediated by specific acid-sensing receptors, four of which are expressed by T cells. Notably, neutralizing tumor acidity with bicarbonate monotherapy impaired the growth of some cancer types in mice where it was associated with increased T-cell infiltration. Furthermore, combining bicarbonate therapy with anti-CTLA-4, anti-PD1, or adoptive T-cell transfer improved antitumor responses in multiple models, including cures in some subjects. In one embodiment of the invention, endothelial progenitor cells, or products thereof, are cultured under conditions in GCN2 kinase is activated, the conditions include culture in the presence of uncharged tRNA, tryptophan deprivation, arginine deprivation, asparagine deprivation [60-64], and glutamine deprivation.

In one embodiment a therapeutic vaccine is provided targeting tumor endothelial cells based on immunization with a composition resembling tumor endothelial cells. Of particular use, the invention discloses the use of tumor endothelial immunization prior to chemotherapy administration in order to reduce chemotherapy associated neovascularization. A recent study demonstrated that adriamycin or paclitaxel, first-line chemotherapy agent, induced breast cancer cells to generate morphological, phenotypical and functional features of endothelial cells in vitro. In xenografts models, challenges from adriamycin or paclitaxel induced cancer cells to generate the majority of microvessels. Importantly, in breast cancer specimens from patients with neoadjuvant anthracycline-based or taxane-based chemotherapy, tumor-derived endothelial microvessels, lined by EGFR-amplified or/and TP53⁺-CD31⁺ endothelial cells, was significantly higher in patients with progressive or stable disease (PD/SD) than in those with a partial or complete response (PR/CR) [67]. In another embodiment, the concurrent use of immunization targeting tumor endothelium together with inhibition of Notch signaling is provided. In another embodiment the concurrent use of VEGFR3 inhibition together with immunization against tumor endothelial cells is provided.

In one specific embodiment, an autologous tumor vaccine is prepared by extraction of tumor derived exosomes as a source of autologous tumor antigens. Specifically, exosomes are purified from circulation using means known in the art. In one particular embodiment, exosomes are isolated from circulation using size exclusion chromatography, such as gel permeation columns, centrifugation or density gradient centrifugation, and filtration methods. For example, exosomes can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used. It is known that highly abundant proteins, such as albumin and immunoglobulin, may hinder isolation of exosomes from a biological sample. For example, exosomes may be isolated from a biological sample using a system that utilizes multiple antibodies that are specific to the most abundant proteins found in blood. Such a system can remove up to several proteins at once, thus unveiling the lower abundance species such as cell-of-origin specific exosomes. This type of system can be used for isolation of exosomes from biological samples such as blood, cerebrospinal fluid or urine. The isolation of exosomes from a biological sample may also be enhanced by high abundant protein removal methods as described in Chromy et al. J. Proteome Res 2004; 3:1120-1127. In another embodiment, the isolation of exosomes from a biological sample may also be enhanced by removing serum proteins using glycopeptide capture as described in Zhang et al, Mol Cell Proteomics 2005; 4:144-155. In addition, exosomes from a biological sample such as urine may be isolated by differential centrifugation followed by contact with antibodies directed to cytoplasmic or anti-cytoplasmic epitopes as described in Pisitkun et al., Proc Natl Acad Sci USA, 2004; 101:13368-13373. Isolation or enrichment of exosomes from biological samples can also be enhanced by use of sonication (for example, by applying ultrasound), or the use of detergents, other membrane-active agents, or any combination thereof. For example, ultrasonic energy can be applied to a potential tumor site, and without being bound by theory, release of exosomes from the tissue can be increased, allowing an enriched population of exosomes that can be analyzed or assessed from a biological sample using one or more methods disclosed herein.

Subsequent to isolation of exosomes from cancer patients, a step of selectivity may be performed to enhance the number of tumor exosomes as compared to healthy exosomes. In some situations, it will not be necessary to isolate out healthy exosomes because of the substantially larger number of tumor exosomes in circulation as compared to healthy exosomes.

It is known that the tumor microenvironment possesses various mechanical properties which effect the tumor cells and tumor endothelial cells, specifically, the extracellular matrix (ECM) around tumors is found to stiffen progressively in a variety of human cancer types. Perhaps the best example is in breast cancer, which is often first detected by direct palpitation due to its increased stiffness compared to surrounding tissues. The presence of a fibrotic focus, which is an accumulation of collagen and fibroblasts, is correlated with an increase in metastatic disease and a decrease in recurrence-free survival in patients.

### Example of Regimen

This regimen is designed for a 3-week protocol with 3 and 6 month follow up from the patient. Blood will be taken for immune analysis at baseline, day 21, month 3 and month 6. Patients are asked to comply with the Gerson diet as a nutritional complement to the regimen. Diagnostic screening and follow up:

1. 50 gene CLEARID liquid biopsy solid tumor panel by Cynvenio

2. PD-L1 liquid biopsy by Cynvenio

3. PGX analysis by autogenomics (as relevant)

4. Circulating tumor cell analysis performed on day 0, day 21, and month 3 and 6 follow up

Weekly cycle repeated 3 times:

### Step 1: Modulation of the Tumor Microenvironment (Starting Day 0)

This step would be comprised of using Immunicom's immunophoresis device to filter out sTNF-A receptor from the blood of cancer patients. The goal is to increase the effective levels of TNF-A within the tumor microenvironment to reduce the effectiveness of the tumor's defense through local immune suppression. This should enhance the effectiveness of subsequent targeted immunotherapies. Patients would undergo the ‘dialysis’ procedure twice a week for approximately 4 hours per session.

### Step 2: Immune Priming (Day 1)

This step would consist of administering GM-CSF 75 mcg subq to locally recruit APC's and to boost white blood cell counts in the patient, priming them for subsequent immunotherapy.

### Step 3: Immune Activation (Day 2)

This step would consist of the administration of Aldara (5% imiquimod) cream 250 mg at the same site of GM-CSF injection, 24 hours following the administration of GM-CSF. Imiquimod is a TLR-4 agonist that is known to induce the maturation of dendritic cells. Here we are leveraging an FDA approved product to ensure that the administration of ValloVax induces a TH1 immune response, without this step there is a change that GM-CSF alone can have an immune suppressive effect.

### Step 4: Active Vaccination (Day 3)

Administration of 10×10\hat{ }6 ValloVax cells at the site of GM-CSF and aldara administration. This step is designed to stimulate an immune response against the vasculature of solid tumors, allowing for increased lymphocyte penetration. This may enhance the effectiveness of other targeted therapies.

### Step 5: Damage to the Tumor (Day 5)

Administration of Stemimmune's vaccinia virus intratumor. The vaccinia virus selectively multiplies within cancer cells while it clears from healthy cells. This would directly damage tumor cells, releasing their antigens into the body through necrosis.

Step 6 (Only on 3rd Cycle): Immune Focusing (this would Only be Applied on the Third Cycle on the Day after Administration of the Oncolytic Virus)/

For this step I would like to leverage the liquid biopsy to identify tumor mutations expressed in the patient. I would like to partner with a personalized vaccine company such as NEO or Gritstone Oncology to gain access to their peptide libraries. What we can do for this step is administer a peptide-based vaccine for mutations expressed in the patient's tumor. This is a step toward further boosting the patient's immune response in an antigen specific manner to induce the generation of Memory T cells.

Step 7 (Only on 3rd Cycle): Immune Boosting (this would be Applied on the Same Day as Immune Focusing on the Third Cycle Only)

Administration of a checkpoint antibody (PD-1) such as Keytruda, if the patient has an expression of PD-L1 within their tumor. The purpose of this step is to sustain the immune response after the patient leaves the clinic.

### Follow Up:

MONTH 3: Patient comes back for a 2-day period. Day 1: administration of GM-CSF and imiquimod simultaneously. Day 2: administration of booster shot of ValloVax. Patient will be required to give blood and imaging (PET-CT)
MONTH 6: Patient comes back for a 2 day period. Day 1: administration of GM-CSF and imiquimod simultaneously. Day 2: administration of booster shot of ValloVax. Patient will be required to give blood and imaging (PET-CT)

1. A method of treating cancer comprising:
a) identifying a cancer patient;
b) assessing genomic, transcriptional profiling, pharmacogenomics, proteomic, metabolic and immunological features of the cancer patient and the cancer mass;
c) treating the cancer patient with an agent or plurality of agents, or devices capable of modulating the tumor microenvironment;
d) priming the immune system so as to prepare the antigen presenting part of the immune system to be on a state of high alert for immune activation;
e) application of an immune activator to induce maturation of antigen presenting cells;
f) administration of a vaccination means to induce immunity towards tumor and/or tumor endothelial cells;
g) induction of immunogenic cell death to the tumor or portion of the tumor;
h) focusing of immune response to antigens correlated with the antigens identified in step “b”; and
i) administration of a boosting means which induces amplification of the immune response.

2. The method of claim 1, wherein the immunological analysis comprises assessment of immunological cells infiltrating the tumor, wherein the immunological cells are selected from a group consisting of: a) monocytes; b) NK cells; c) NKT cells; d) T cells; e) B cells; f) gamma delta T cells; and g) neutrophils.

3. The method of claim 1, wherein the immunological analysis comprises assessment of immunological cells in circulation, wherein the immunological cells are selected from a group consisting of: a) monocytes; b) NK cells; c) NKT cells; d) T cells; e) B cells; f) gamma delta T cells; and g) neutrophils.

4. The method of claim 3, wherein the immunological analysis comprises assessment of immunological activity of the cells in circulation, wherein the immunological activity is at least one of secretion of cytokines, cytotoxic activity, expression of costimulatory molecules, and production of angiogenic inhibitory factors.

5. The method of claim 4, wherein the cytokines are selected from a group consisting of: a) IL-1; b) IL-2; c) IL-4; d) IL-7; e) IL-10; f) IL-12; g) IL-15; h) IL-18; i) IL-20; j) IL-23; k) IL-25; l) IL-27; m) IL-33; n) IFN-alpha; o) IFN-gamma; and p) TGF-beta.

6. The method of claim 1, wherein the modulation of the tumor microenvironment is achieved by immunopheresis to remove tumor produced immune inhibitory molecules.

7. The method of claim 6, wherein the tumor produced immune inhibitory molecule is selected from the group consisting of VEGF, TGF-beta, PGE-2, and non-cytotoxic antibodies.

8. The method of claim 1, wherein the modulation of the tumor microenvironment is achieved by administration of an inhibitor of indolamine 2,3 deoxygenase.

9. The method of claim 8, wherein the inhibitor of indolamine 2,3 deoxygenase is selected from the group consisting of an antisense oligonucleotide molecule, a hammerhead ribozyme, a short interfering RNA interfering molecule, a short hairpin RNA interfering molecule, a small molecule inhibitor, a 1-methyltryptophan, a heme precursor compound zinc protoporphyrin IX, hydroxyamidine, Withaferin A, Ferulic acid, scabanol (2), lavender oil, Astragaloside IV, galanal, and curcumin.

10. The method of claim 1, wherein modulation of the tumor microenvironment is performed by PGE-2 inhibition.

11. The method of claim 10, wherein the PGE-2 inhibition is performed by administration of at least one of celecoxib, ibuprofen, indomethacin, and nimesulide.

12. The method of claim 1, wherein modulation of the tumor microenvironment is performed by reduction of myeloid suppressor cells.

13. The method of claim 12, wherein the reduction of myeloid suppressor cells is achieved by administration of at least one of vitamin D3, a PDE-5 inhibitor, and a VEGF or VEGF receptor inhibitor.

14. The method of claim 1, wherein modulation of the local tumor environment is accomplished by reduction of T regulatory cells.

15. The method of claim 1, wherein the priming of the immune system is achieved by administration of at least one of GM-CSF, FLT-3L, and Mozibil.

16. The method of claim 1, wherein the immune activator is an inducer of dendritic cell maturation.

17. The method of claim 16, wherein the inducer of dendritic cell maturation is a toll like receptor agonist, wherein the toll like receptor is selected from the group consisting of TLR-2, TLR-3, TLR-4, TLR-5, TLR-7, TLR-8, and TLR-9.

18. The method of claim 1, wherein the vaccination is performed in order to induce immunity to tumor endothelial cells by immunization with placental endothelial cells.

19. The method of claim 18, wherein the immunization with placental endothelial cells is performed through the use of ValloVax administration.

20. The method of claim 18, wherein the immunization with placental endothelial cells is performed using administration of endothelial cells grown in a manner to induce a tumor endothelial-like phenotype.
